(19) United States
(12) Patent Application Publication
(10) Pub. No.: US 2014/0155840 A1
(43) Pub. Date: Jun. 5, 2014

(54) Medical cream applying apparatus

(71) Applicants: Susan Tseng, West Covina, CA (US); Ya-Zhou Ma, Rosemead, CA (US)

(72) Inventors: Susan Tseng, West Covina, CA (US); Ya-Zhou Ma, Rosemead, CA (US)

(21) Appl. No.: 13/694,464

(22) Filed: Dec. 3, 2012

(51) Int. Cl. A61M 31/00

(52) U.S. Cl. CPC A61M 31/00
USPC 604/257; 604/279

(57) ABSTRACT

A medical cream applying apparatus includes an internal cream applicator which includes a handle member and an inserting head member. The handle member is ergonomically designed to allow a user's hand to grab thereon. The inserting head member is extended from the handle member, and has a round inserting portion having a diameter slightly larger than that of the handle member, wherein the round inserting portion is adapted to be soaked with a predetermined amount of medical cream, and is arranged to slightly insert into a vagina of a female user for evenly applying the medical cream in the vagina for treating a predetermined disease.

### BACKGROUND OF THE PRESENT INVENTION

1. Field of Invention

The present invention relates to a medical cream applying apparatus, and more particularly to an internal cream applicator which is arranged to be inserted into a vagina of a female user so as to evenly apply medical cream thereto.

2. Description of Related Arts

A conventional method for applying a medical cream, such as antifungal cream, into a female's vagina is to use a syringe. A user needs to put a predetermined amount of medical cream into the syringe's barrel. After that, the user needs to insert the contracted portion of the barrel into her vagina and pushes the plunger of the syringe so as to eject the medical cream into the vagina. After the process, the syringe is removed from the vagina and should be disposed.

There are several disadvantages in relation to this conventional practice. First, the medical cream applied by the above-mentioned method will not be uniform in the user's vagina. Strictly speaking, the above method does not “apply” medical cream into the user's vagina. Rather, the syringe merely injects a predetermined amount of medical cream into the user's vagina without evenly applying the medical cream in the surrounding lining or tissue of the user's vagina. Thus, the user does not know if the medical cream is actually applied onto the affected area, and this substantially undermine the effect of treating the medical condition in the user's vagina.

Moreover, it is very inconvenient to suck the medical cream into the syringe's barrel. The container which originally stores the medical cream must be specifically designed for allowing the medical cream to be sucked into a syringe. When the container is not designed to allow such procedure, it is very inconvenient for the user to put the medical cream into the syringe. Furthermore, after the syringe is used for ejecting the medical cream, it is very inconvenient for the user to clean the syringe. As a result, the syringe is usually disposed and this leads to substantial waste of medical resources.

### SUMMARY OF THE PRESENT INVENTION

The invention is advantageous in that it provides a medical cream applying apparatus comprising an internal cream applicator which is arranged to be inserted into a vagina of a female user so as to evenly apply medical cream thereto.

Another advantage of the invention is to provide a medical cream applying apparatus comprising an internal cream applicator which comprises a handle member and an inserting head member arranged to be soaked with a predetermined amount of medical cream, wherein the inserting head member is to be inserted into the user's vagina for accurately applying the medical cream onto the affected area.

Another advantage of the invention is to provide a medical cream applying apparatus comprising an internal cream applicator, wherein the inserting head member has a round inserting portion which is capable of evenly applying the medical cream onto an inner lining tissue of the user's vagina.

Another advantage of the invention is to provide a medical cream applying apparatus comprising a cream applying device which is specifically designed for soaking a predetermined amount of medical cream onto the internal cream applicator so as to allow a user to conveniently use the medical cream for treating her vaginal condition.

Additional advantages and features of the invention will become apparent from the description which follows, and may be realized by means of the instrumentalities and combinations particular point out in the appended claims.

According to the present invention, the foregoing and other objects and advantages are attained by providing a medical cream applying apparatus which comprises an internal cream applicator, comprising:

a handle member which is ergonomically designed to allow a user's hand to grab thereon; and

an inserting head member extended from the handle member, wherein the inserting head member has a round inserting portion having a diameter slightly larger than that of the handle member, and is adapted to be soaked with a predetermined amount of medical cream, wherein the round inserting portion of the inserting head member is arranged to be slightly inserted into a vagina of a female user for evenly applying the medical cream in the vagina for treating a predetermined disease.

Still further objects and advantages will become apparent from a consideration of the ensuing description and drawings.

These and other objectives, features, and advantages of the present invention will become apparent from the following detailed description, the accompanying drawings, and the appended claims.

### BRIEF DESCRIPTION OF THE DRAWINGS

FIG. 1 is a perspective view of an internal cream applicator according to a preferred embodiment of the present invention.

FIG. 2 is a schematic view of a medical cream applying device according to the above preferred embodiment of the present invention.

FIG. 3 a first alternative mode of the medical cream applying apparatus according to the above preferred embodiment of the present invention.

FIG. 4 a second alternative mode of the medical cream applying apparatus according to the above preferred embodiment of the present invention.

### DETAILED DESCRIPTION OF THE PREFERRED EMBODIMENT

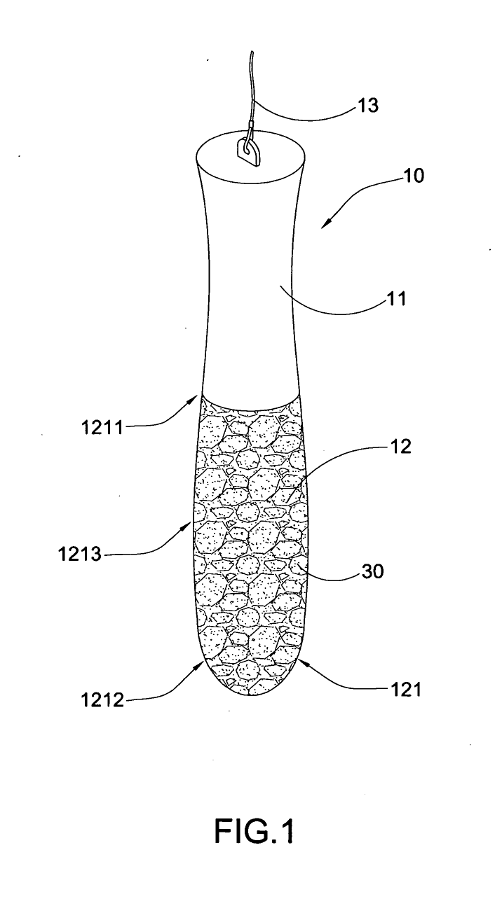

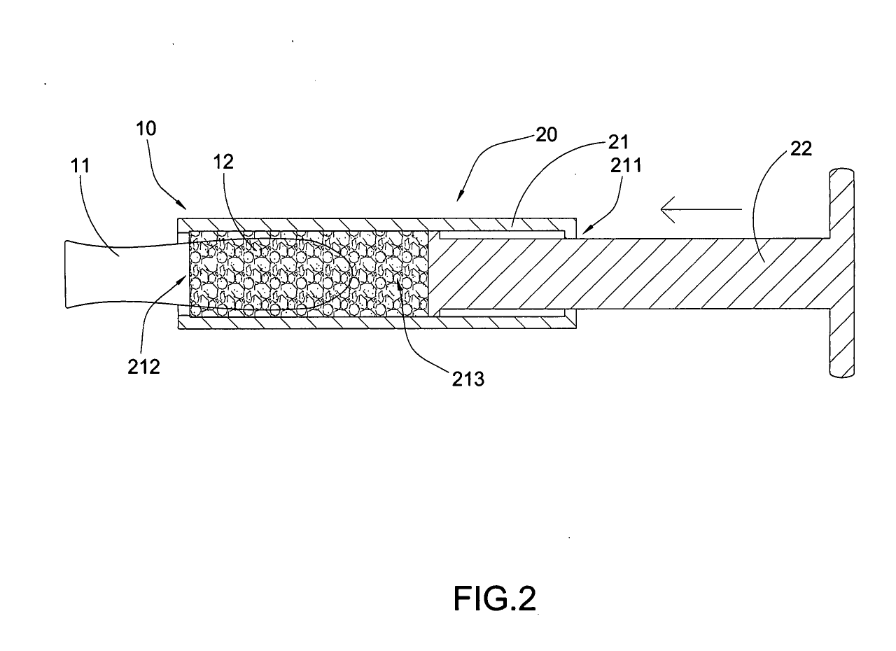

Referring to FIG. 1 to FIG. 2 of the drawings, a medical cream applying apparatus according to a preferred embodiment of the present invention is illustrated, in which the medical cream applying apparatus comprises an internal cream applicator 10, which comprises a handle member 11 and an inserting head member 12.

The handle member 11 is elongated in shape and is ergonomically designed to allow a user's hand to grab thereon.

The inserting head member 12 is extended from the handle member 11, wherein the inserting head member 12 has a round inserting portion 121 having a diameter slightly larger than that of the handle member 11, and is adapted to be soaked with a predetermined amount of medical cream 30, wherein the round inserting portion 121 of the inserting head member 12 is arranged to be slightly inserted into a vagina of a female user for evenly applying the medical cream 30 in the vagina for treating a predetermined disease.

According to the preferred embodiment of the present invention, the medical cream applying apparatus further comprises a cream applying device 20 which is arranged to store a predetermined amount of medical cream therein, and is arranged to operate with the internal cream applicator for soaking the medical cream onto the round inserting portion 121 of the inserting head member 12. In other words, the cream applying device and the internal cream applicator 10 form a medical kit for allowing a user to apply a predetermined amount of medical cream into her vagina for treating the corresponding vaginal disease.

The handle member 11 of the internal cream applicator 10 has a length which is longer than a width of a user's hand so that the user's hand is able to grab thereon. The handle member 11 and the inserting head member 12 may be integrally connected to form a single integral body, or they can be separate members each of which can be detachable from each other. In this preferred embodiment, the handle member 11 and the inserting head member 12 are preferably integrally extended from each other. The exemplary embodiments in which the handle member 11 and the inserting head member 12 are separate members will be described below.

The inserting head member 12 is ergonomically designed to be inserted into a female's vagina so that the cream applied on the round inserting portion 121 can be evenly applied to the side walls or inner lining of the vagina. It is worth mentioning that by applying the medical cream through the round inserting portion 121, the medical cream can be evenly applied to the side wall or inner lining of the user's vagina and this substantially resolves the problems mentioned for conventional prior practices (mentioned previously).

As shown in FIG. 1 of the drawings, the round inserting portion 121 of the inserting head member 12 has varying diameters along a longitudinal direction thereof. More specifically, the round inserting portion 121 has an rear section 1211, a front tip section 1212, and a mid section 1213 extended between the rear section 1211 and the front tip section 1212, wherein a cross sectional diameter of the mid section 1213 is greater than that of the rear section 1211 and the front tip section 1212. This design allows the inserting head member 12 to be easily inserted into the user's vagina, and while the inserting head member 12 is inserted into the user's vagina, the relatively enlarged mid section 1213 of the round inserting portion 121 allows the medical cream to be easily and evenly applied to the inner side wall or lining tissue of the user's vagina.

In order to allow easy carrying or storage, the internal cream applicator 10 further comprises means for attaching the handle member 11 and the inserting head member 12 onto an external object. In this preferred embodiment, the attaching means comprises a soft or elastic attaching member 13, such as an attaching rope, extended from a rear portion of the handle member 11 for detachably attaching on an external object.

Referring to FIG. 2 of the drawings, the medical cream applying apparatus further comprises cream applying device 20 used in conjunction of the internal cream applicator 10 for applying the medical cream onto the internal cream applicator 10 so that the user may in turn insert the inserting head member 12 into her vagina for applying the medical cream thereon.

The cream applying device 20 comprises an applying barrel 21 having a plunger opening 211 formed at one end thereof, an applicator opening 212 formed opposite to the plunger opening 211 at another end of the applying barrel 21, and a receiving cavity 213 formed between the plunger opening 211 and the applicator opening 212 in the applying barrel 21. The inserting head member 12 of the internal cream applicator 10 is arranged to detachably insert into the receiving cavity 213 through the applicator opening 212.

The cream applying device 20 further comprises a plunger device 22 slidably and detachably mounted at the plunger opening 211, wherein a predetermined amount of medical cream is filled in the receiving cavity 213 near the plunger opening 211 so that when the plunger device 22 is pushed toward the applicator opening 212, the medical cream is pushed by the plunger device 22 to apply onto the round inserting portion 121 of the inserting head member 12.

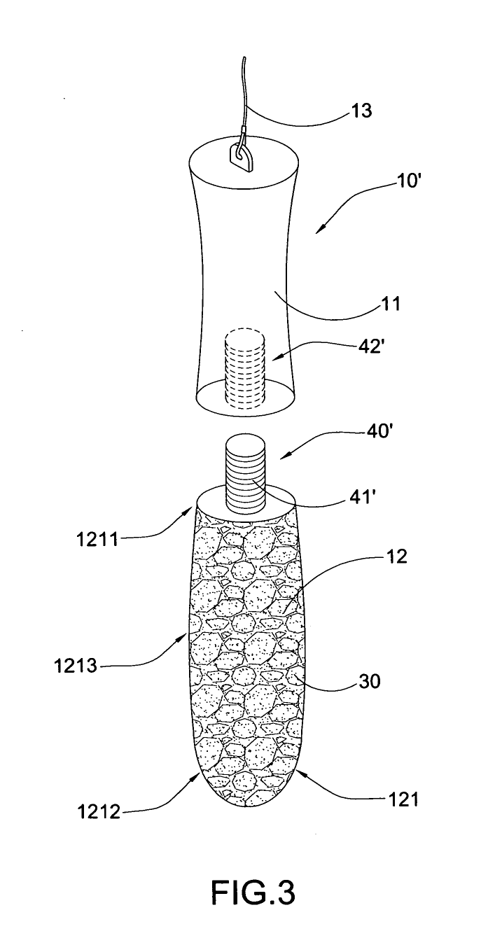

Referring to FIG. 3 of the drawings, a first alternative mode of the medical cream applying apparatus according to the preferred embodiment of the present invention is illustrated. The first alternative mode is similar to the preferred embodiment, except the internal cream applicator 10′. In this first alternative mode, the internal cream applicator 10′ further comprises a detachable arrangement 40′ provided on the handle member 11 and the inserting head member 12 for detachably connecting them together. More specifically, the detachable arrangement 40′ comprises a connector member 41′ provided on one of the handle member 11 and the inserting head member 12, and contains a connecting slot 42′ provided on the other of the other one of the handle member 11 and the inserting head member 12, wherein the connector member 41′ is arranged to detachably couple with the connecting slot 42′ so as to detachably connect the handle member 11 with the inserting head member 12.

In this first alternative mode, the connecting member 41′ is preferably embodied as a threaded pin extended from a front end of the handle member 11, while the connecting slot 42′ is preferably embodied as bored hole formed on a rear end of the inserting head member 12, wherein the connecting member 41′ is arranged to threadedly couple with the connecting slot 42′ for detachably connecting the handle member 11 with the inserting head member 12.

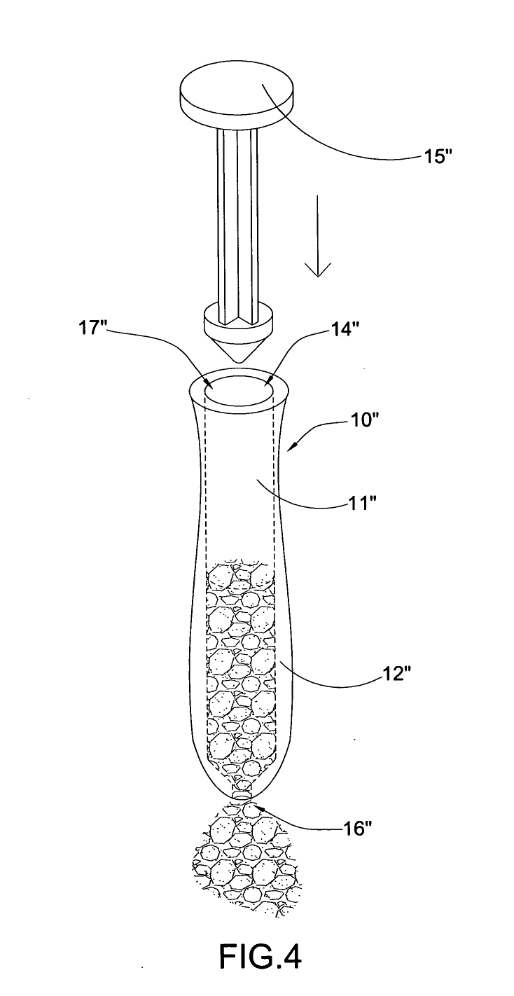

Referring to FIG. 4 of the drawings, a second alternative mode of the medical cream applying apparatus according to the preferred embodiment of the present invention is illustrated. The second alternative mode is similar to the preferred embodiment, except the internal cream applicator 10″. According to the second alternative mode, the internal cream applicator 10″ further has a storing cavity 14″ formed therein, a through passage hole 16″ formed a front end of the inserting head member 12″, and through a pusher hole 17″ formed on a rear end of the handle member 11″, wherein the passage hole 16″ and the pusher hole 17″ communicate with the storing cavity 14″.

Moreover, the internal cream applicator 10″ further comprises a pusher member 15″ slidably mounted at the pusher hole 17″, and is arranged to slidably move along a longitudinal direction of the internal cream applicator 10″ in the storing cavity 14″ so that when the medical cream is disposed in the storing cavity 14″, a pushing of the pusher hole 17″ will result in the medical cream being ejected out of the internal cream applicator 10″ through the passage hole 16″.

In this second alternative mode, the handle member 11″ and the inserting head member 12′ form an integral body.

One skilled in the art will understand that the embodiment of the present invention as shown in the drawings and described above is exemplary only and not intended to be limiting.

It will thus be seen that the objects of the present invention have been fully and effectively accomplished. It embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A medical cream applying apparatus for female vagina which comprises an internal cream applicator, comprising:
a handle member which is ergonomically designed to allow a user's hand to grab thereon; and
an inserting head member extended from said handle member, wherein said inserting head member has a round inserting portion having a diameter slightly larger than that of said handle member, and is adapted to be soaked with a predetermined amount of medical cream, wherein said round inserting portion of said inserting head member is arranged to be slightly inserted into a vagina of a female user for evenly applying said medical cream in said vagina for treating a predetermined disease.

2. The medical cream applying apparatus, as recited in claim 1, further comprising a cream applying device which is arranged to store a predetermined amount of medical cream therein, and is arranged to operate with said internal cream applicator for soaking said medical cream onto said round inserting portion of said inserting head member.

3. The medical cream applying apparatus, as recited in claim 1, wherein said handle member of said internal cream applicator has a length which is longer than a width of a user's hand so that said user's hand is able to grab thereon.

4. The medical cream applying apparatus, as recited in claim 2, wherein said handle member of said internal cream applicator has a length which is longer than a width of a user's hand so that said user's hand is able to grab thereon.

5. The medical cream applying apparatus, as recited in claim 3, wherein said handle member and said inserting head member is integrally connected to form a single integral body.

6. The medical cream applying apparatus, as recited in claim 4, wherein said handle member and said inserting head member is integrally connected to form a single integral body.

7. The medical cream applying apparatus, as recited in claim 5, wherein said round inserting portion has a rear section, a front tip section, and a mid section extended between said rear section and said front tip section, wherein a cross sectional diameter of said mid section is greater than that of said rear section and said front tip section.

8. The medical cream applying apparatus, as recited in claim 6, wherein said round inserting portion has a rear section, a front tip section, and a mid section extended between said rear section and said front tip section, wherein a cross sectional diameter of said mid section is greater than that of said rear section and said front tip section.

9. The medical cream applying apparatus, as recited in claim 7, wherein said internal cream applicator further comprises means for detachably attaching said internal cream applicator onto an external object.

10. The medical cream applying apparatus, as recited in claim 8, wherein said internal cream applicator further comprises means for detachably attaching said internal cream applicator onto an external object.

11. The medical cream applying apparatus, as recited in claim 2, wherein said cream applying device comprises an applying barrel having a plunger opening formed at one end thereof, an applicator opening formed opposite to said plunger opening at another end of said applying barrel, and a receiving cavity formed between said plunger opening and said applicator opening in said applying barrel, wherein said inserting head member of said internal cream applicator is arranged to detachably insert into said receiving cavity through said applicator opening.

12. The medical cream applying apparatus, as recited in claim 9, wherein said cream applying device comprises an applying barrel having a plunger opening formed at one end thereof, an applicator opening formed opposite to said plunger opening at another end of said applying barrel, and a receiving cavity formed between said plunger opening and said applicator opening in said applying barrel, wherein said inserting head member of said internal cream applicator is arranged to detachably insert into said receiving cavity through said applicator opening.

13. The medical cream applying apparatus, as recited in claim 11, wherein said cream applying device further comprises a plunger device slidably and detachably mounted at said plunger opening, wherein a predetermined amount of medical cream is filled in said receiving cavity near said plunger opening so that when said plunger device is pushed toward said applicator opening, said medical cream is pushed by said plunger device to apply onto said round inserting portion of said inserting head member.

14. The medical cream applying apparatus, as recited in claim 12, wherein said cream applying device further comprises a plunger device slidably and detachably mounted at said plunger opening, wherein a predetermined amount of medical cream is filled in said receiving cavity near said plunger opening so that when said plunger device is pushed toward said applicator opening, said medical cream is pushed by said plunger device to apply onto said round inserting portion of said inserting head member.

15. The medical cream applying apparatus, as recited in claim 8, wherein said internal cream applicator further comprises a detachable arrangement which comprises a connector member provided on one of said handle member and said inserting head member, and contains a connecting slot provided on said other of said other one of said handle member and said inserting head member, wherein said connector member is arranged to detachably couple with said connecting slot so as to detachably connect said handle member with said inserting head member.

16. The medical cream applying apparatus, as recited in claim 10, wherein said internal cream applicator further comprises a detachable arrangement which comprises a connector member provided on one of said handle member and said inserting head member, and contains a connecting slot provided on said other of said other one of said handle member and said inserting head member, wherein said connector member is arranged to detachably couple with said connecting slot so as to detachably connect said handle member with said inserting head member.

17. The medical cream applying apparatus, as recited in claim 15, wherein said internal cream applicator further comprises a detachable arrangement which comprises a connector member provided on one of said handle member and said inserting head member, and contains a connecting slot provided on said other of said other one of said handle member and said inserting head member, wherein said connector member is arranged to detachably couple with said connecting slot so as to detachably connect said handle member with said inserting head member.

18. The medical cream applying apparatus, as recited in claim 8, wherein said internal cream applicator further has a storing cavity formed therein, a through passage hole formed a front end of said inserting head member, and through a pusher hole formed on a rear end of said handle member, wherein said passage hole and said pusher hole communicate with said storing cavity.

19. The medical cream applying apparatus, as recited in claim 10, wherein said internal cream applicator further has a storing cavity formed therein, a through passage hole formed a front end of said inserting head member, and through a pusher hole formed on a rear end of said handle member, wherein said passage hole and said pusher hole communicate with said storing cavity.

20. The medical cream applying apparatus, as recited in claim 15, wherein said internal cream applicator further has a storing cavity formed therein, a through passage hole formed a front end of said inserting head member, and through a pusher hole formed on a rear end of said handle member, wherein said passage hole and said pusher hole communicate with said storing cavity.

21. The medical cream applying apparatus, as recited in claim 18, wherein said internal cream applicator further comprises a pusher member slidably mounted at said pusher hole, and is arranged to slidably move along a longitudinal direction of said internal cream applicator in said storing cavity so that when said medical cream is disposed in said storing cavity, a pushing of said pusher hole results in said medical cream being ejected out of said internal cream applicator through said passage hole.

22. The medical cream applying apparatus, as recited in claim 19, wherein said internal cream applicator further comprises a pusher member slidably mounted at said pusher hole, and is arranged to slidably move along a longitudinal direction of said internal cream applicator in said storing cavity so that when said medical cream is disposed in said storing cavity, a pushing of said pusher hole results in said medical cream being ejected out of said internal cream applicator through said passage hole.

23. The medical cream applying apparatus, as recited in claim 20, wherein said internal cream applicator further comprises a pusher member slidably mounted at said pusher hole, and is arranged to slidably move along a longitudinal direction of said internal cream applicator in said storing cavity so that when said medical cream is disposed in said storing cavity, a pushing of said pusher hole results in said medical cream being ejected out of said internal cream applicator through said passage hole.
